(19) United States
(12) Patent Application Publication
(10) Pub. No.: US 2026/0256394 A1
(43) Pub. Date: Sep. 3, 2026

(54) A BABY MONITORING SYSTEM AND METHOD

(71) Applicant: KONINKLIJKE PHILIPS N.V., EINDHOVEN (NL)

(72) Inventors: DAAN KUHLMANN, HEESCH (NL); JOZEF HUBERTUS GELISSEN, HERTEN (NL)

(21) Appl. No.: 18/871,576

(22) PCT Filed: Jun. 5, 2023
(86) PCT No.: PCT/EP2023/064905

§ 371 Date: Dec. 4, 2024

(30) Foreign Application Priority Data
Jun. 14, 2022 (EP) 22178936.5

(51) Int. Cl. A61B 5/16; A61B 5/00; A61B 5/0205; A61B 5/024; A61B 5/0533; A61B 5/08; G06T 7/00

(52) U.S. Cl. CPC A61B 5/165; A61B 5/0205; A61B 5/024; A61B 5/0533; A61B 5/0816; A61B 5/7235; A61B 5/7282; A61B 5/7405; A61B 5/742; G06T 7/0012; A61B 2503/04; A61B 2576/02; G06T 2207/10016; G06T 2207/30201

(57) ABSTRACT

A baby monitoring system includes a monitoring unit for monitoring a parent emotional state (or for receiving information concerning a parent emotional state from a remote monitoring unit). The baby monitoring system generates an output which depends on the parent emotional state.

### FIELD OF THE INVENTION

This invention relates to baby monitoring systems, namely systems with a parent unit and a baby unit, enabling a parent to hear, and optionally also see (in the case of a video baby monitor system) what the baby is doing.

### BACKGROUND OF THE INVENTION

Baby monitoring systems are traditionally focused on the wellbeing of the baby, for example triggering a parent unit to turn on when the baby makes a sound.

Many women experience symptoms of anxiety in the later parts of pregnancy or in initial postnatal period after the birth. Both parents can suffer from several emotional effects: loneliness, bonding worries and even guilt. One in five parents (especially mothers) develop one or more of those effects after having a baby. If left unchecked, these can lead to serious stress or depression, which in turn is also bad for the baby wellbeing and bonding with parents. The emotional state of one parent will also have an impact on the emotional state of the other parent.

The first step in remedying these problems is to acknowledge that the problem is present. Thus, it would be of interest to provide information to a parent to identify that there is a potential emotional issue which could be addressed.

EP 3 522 170 discloses a system for alerting a subject, of a plurality of possible subjects, to an event. The subject is chosen based on measured vital parameters of the plurality of possible subjects.

U.S. Pat. No. 9,538,959 discloses a monitoring system for use with multiple caregivers. Vital data and sleep data are collected for the caregivers and used to determine which caregiver should be provided with an alarm when the subject being monitored (e.g. a sleeping baby) wakes up.

U.S. Pat. No. 9,811,992 discloses a system alerting a caregiver on a ward, based on the capacity of the caregiver to respond to the needs of the ward.

### SUMMARY OF THE INVENTION

The invention is defined by the claims.

According to examples in accordance with an aspect of the invention, there is provided a baby monitoring system, comprising:
a baby unit comprising at least a microphone for picking up baby sounds and a baby unit communications system;
a parent unit comprising at least a speaker and a parent unit communications system, wherein the speaker is for outputting baby sounds transmitted from the baby unit to the parent unit using the communications systems;
a monitoring unit for monitoring a parent emotional state or an input device for receiving information concerning a parent emotional state from a remote monitoring unit; and
an output unit for generating an output which depends on the parent emotional state.

The monitoring unit or remote monitoring unit comprises one or more of a galvanic skin response sensor, a heart rate sensor, a respiration rate sensor, a motion sensor and a camera. This system incorporates emotional monitoring into a baby monitor system thereby extending the functionality to include monitoring the wellbeing of the parent as well as the baby. The monitoring system may be integrated into the parent unit of the baby monitoring system or it may be an additional extension unit, or it may be implemented by other on-person hardware (smartwatch, smartphone) already used by the parent that can check the emotional wellbeing of the parent. The output system is part of the baby monitoring system, for example it is controlled by the parent unit. It for example sends a message as feedback to the parent or parents or even to others (e.g. by using an interface to the Internet) to request or recommend that they provide help. In this way, the wellbeing of both the parents and the baby are monitored so the negative effects of having a newborn can be detected in a timely manner.

The system for example comprises a video baby monitoring system, wherein the baby unit comprises a camera and the parent unit comprises a display. Thus, the invention may be applied to a video baby monitoring system.

The system comprises a trigger system for triggering the monitoring unit to obtain a parent emotional state when:
the baby unit triggers the parent unit to activate in order to alert the parent to baby activity; or
the parent unit is detected as being in use.

The parent unit is for example detected as being in use if it being held by the parent, and is moving. This saves energy in that the emotional monitoring only takes place when the parent unit is in use. This is a time when a parent may be most anxious as they are remote from their baby and they will be monitoring the output of the parent unit, so that messages provided to the parent unit are less likely to be unnoticed or ignored.

The monitoring unit for example comprises a galvanic skin response sensor. The sweat response is well known to be a stress indicator.

The monitoring unit may comprise a heart rate sensor or a respiration rate sensor. The heart rate and respiration rate are also well known to be stress indicators.

For these purposes, the monitoring unit for example comprises a dry electrode sensor. It can be used for galvanic skin response and heart rate monitoring.

The monitoring unit may comprise a camera for capturing images of the parent. Facial expressions may for example assist in determining an emotional state.

An image processing module for example is used for processing images from the camera to detect one or more of:
excessive looking at a video of the baby; and
an angry or irritated facial expression.

These may be signs of emotional anguish.

The monitoring unit for example comprises a motion sensor such as an accelerometer. A motion processing module may then process data from the motion sensor for example to detect parent shaking or trembling.

The output unit may be configured to:
provide feedback to the parent;
send a communication to a relative, friend, or maternity carer.

Thus, the system is used to provide advice to the parent or relative/friend to make them aware of the possible emotional condition, which can then be addressed.

The invention also provides a baby monitoring method, comprising:
picking up baby sounds at a baby unit of a baby monitoring system;
transmitting baby sounds from the baby unit to a parent unit;
outputting the baby sounds at the parent unit;
monitoring a parent emotional state; and
generating an output which depends on the parent emotional state.

The monitoring of the parent emotional state uses one or more of a galvanic skin response sensor, a heart rate sensor, a respiration rate sensor, a motion sensor and a camera.

The monitoring of a parent emotional state is triggered when:
the baby unit triggers the parent unit to activate to alert the parent to baby activity; or
the parent unit is detected as being in use.

The parent emotional state is for example monitored by one or more of:
monitoring galvanic skin response;
monitoring heart rate;
processing images captured images of the parent;
monitoring movement of the parent.

The invention also provides a computer program comprising computer program code which is adapted, when said program is run on a processor of the system defined above to implement the method defined above.

These and other aspects of the invention will be apparent from and elucidated with reference to the embodiment(s) described hereinafter.

### BRIEF DESCRIPTION OF THE DRAWINGS

For a better understanding of the invention, and to show more clearly how it may be carried into effect, reference will now be made, by way of example only, to the accompanying drawings, in which:

FIG. 1 shows a baby monitoring system;

FIG. 2 shows the components of the baby monitoring system in more detail;

FIG. 3 shows a method of interpreting sensor data which depends on an emotional state; and

FIG. 4 shows an example of a plot of the emotional state of a parent over time.

### DETAILED DESCRIPTION OF THE EMBODIMENTS

The invention will be described with reference to the Figures.

It should be understood that the detailed description and specific examples, while indicating exemplary embodiments of the apparatus, systems and methods, are intended for purposes of illustration only and are not intended to limit the scope of the invention. These and other features, aspects, and advantages of the apparatus, systems and methods of the present invention will become better understood from the following description, appended claims, and accompanying drawings. It should be understood that the Figures are merely schematic and are not drawn to scale. It should also be understood that the same reference numerals are used throughout the Figures to indicate the same or similar parts.

The invention provides a baby monitoring system includes a monitoring unit for monitoring a parent emotional state (or for receiving information concerning a parent emotional state from a remote monitoring unit). The baby monitoring system generates an output which depends on the parent emotional state. The output “depends on” the emotional state in the sense that the output conveys different information for different emotional states or it may be sent to a different recipient for different emotional states. The output may “depend on” the emotional state simply because it identifies the emotional state itself, but it may instead perform an action which is a function of the emotional state without actually identifying the emotional state (e.g. score) itself.

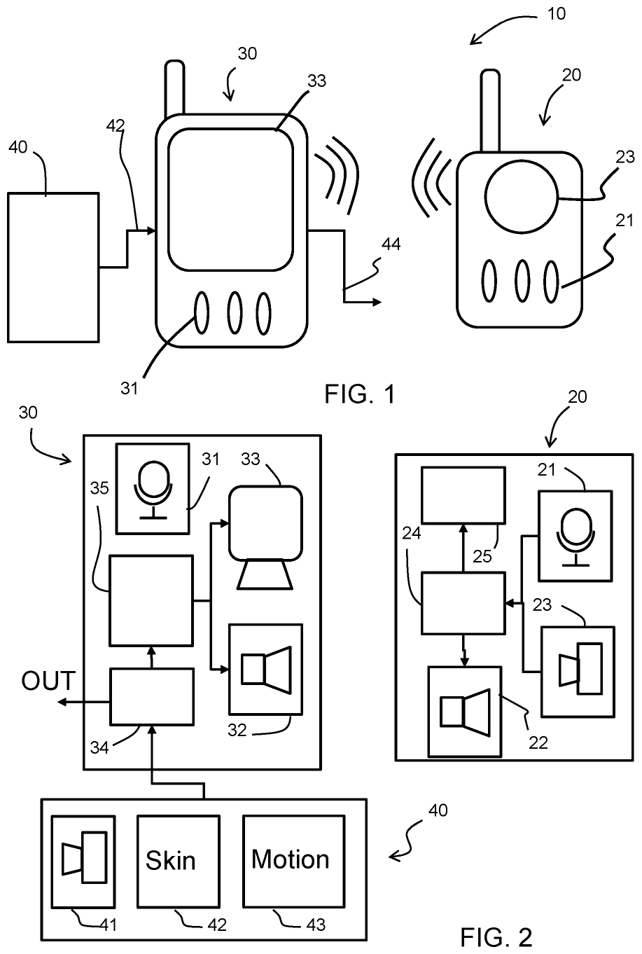

FIG. 1 shows a baby monitoring system 10, comprising a baby unit 20 and a parent unit 30. The baby unit is placed near the baby to pick up at least sounds made by the baby using microphone 21. The parent can listen to the baby sound using the parent unit 30, or the parent unit may be silent unless sounds are made which exceed a threshold. In a more advanced system, the baby unit may interpret the sounds made by the baby and alert the parent via the parent unit if particular sounds are made, e.g. crying, coughing choking etc.

The baby unit is usually stationary as the baby (from infancy to around age 2 years) typically spends most of their time while being monitored in bed. The parent unit will however be carried around by the parents, while they participate in different activities around the home. Therefore, the parent unit is usually designed to have a built in battery so that the system is still operable while the parents carry out different activities.

The baby unit and the parent unit communicate with each other, for example using a 2.4 GHz ISM band wireless transceiver with a proprietary communication protocol such as Frequency Hopping Spread Spectrum (FHSS) for example for transmitting audiovisual data. The general characteristic of such technology is that the device has a high transmitting power, high receiving sensitivity and has a low data rate. The baby monitor can function over a relatively long range such as 300 m, and with low cost.

It has been proposed for the baby unit and parent unit to be able to connect to the internet. For example, the system can operate in a local communication session in a peer-to-peer mode over a local network or in a remote mode over a wide area network. WiFi modules may be provided in the baby unit and the parent unit to enable communication with each other, as well as with other devices, over the internet.

The baby unit may more generally be considered to be a monitor unit, and the parent unit may more generally be considered to be a receiving unit. The parent unit may be considered to be a receiving unit because the primary purpose is to receive and output sound (and optionally images) that have been received from the baby unit. However, the system typically allows two way audio communication, so that the parent unit can also be used to transmit audio to the baby unit. Thus, the parent unit can also be used as a transmitter.

The system shown in FIG. 1 comprises a remote monitoring unit 40 for monitoring a parent emotional state. This monitoring unit may instead be an integral part of the system, but in the system of FIG. 1, the parent unit has an input 42 for receiving information concerning a parent emotional state from the remote monitoring unit 40.

The system, and in particular the parent unit 30, has an output unit for generating an output 44 which depends on the parent emotional state.

FIG. 2 shows the components of the system in more detail. The baby unit 20 comprises the microphone 21 as mentioned above, and in this more advanced example it also comprises a speaker 22, an image sensor 23 (e.g. a digital camera), and an image and audio processor 24. The microphone 21 and image sensor 23 are examples of input device. The baby unit may have only input devices, but this example also includes an output device in the form of the speaker 22. The processed image and audio is transmitted by a communications system, in the form of a transceiver 25. It may for example implement a frequency hopping spread spectrum (FHSS) modulation. The modulated signal is transmitted on the 2.4 GHz band using an antenna.

The parent unit 30 comprises a microphone 31, a speaker 32, a display 33 (e.g. a LCD screen), and an image and audio processor 34. The speaker and display are examples of output device. The parent unit may have only output devices, but this example also includes an input device in the form of the microphone 31. The parent unit has a communications system 35, again in the form of a transceiver for communicating with the baby unit.

FIG. 2 also shows various possible options for the monitoring unit 40, which may be used individually or in combination.

One option is a camera 41, for capturing images of the parent. The processor 35 of the parent unit may then include an image processing module for processing the images from the camera to detect an emotional state of the parent.

Another option is a galvanic skin response sensor 42 for measuring skin conductivity. Skin conductivity sensing may also be used for monitoring heart rate in a similar manner to an ECG. For example, a dry electrode sensor may be used.

The same sensing may be used to detect respiration rate. Alternatively, respiration rate may be obtained by remoted PPG sensing based on image sensing.

Another option is a motion sensor 43. The processor 35 of the parent unit may then include a motion processing module for processing data from the motion sensor, for example to detect parent shaking or trembling.

The remote monitoring unit 40 may comprise a user's smartwatch or smartphone which includes vital signs and/or motion sensing. The monitoring data may however be collected from both a remote device or devices and from local sensors.

The processor 35 in the parent unit collects the various monitoring information and converts the data into a single value or set of values representing an emotional state or states of the parent. For example, there may be a score of 0 to 10 which indicates the urgency with which help is needed, derived from the psychological or emotional state of the parent. For instance, if multiple indications are sensed, the emotional state score will be much higher than when only one indicator is sensed.

Also, the type of response that has been sensed may be weighted, for example an angry facial expression may have a higher weighted score than an increased respiration rate.

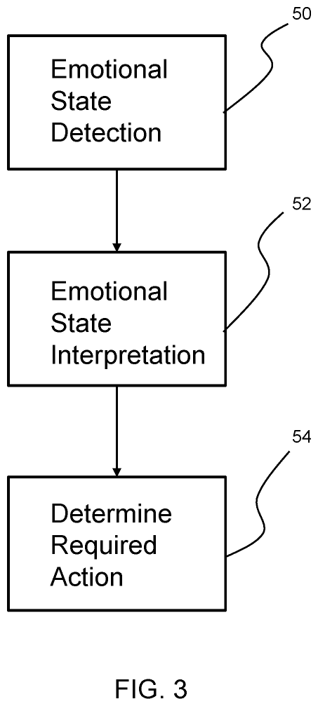

FIG. 3 shows the processing of the sensor data. In step 50, the sensed monitoring data is collected, which is dependent on the emotion state. The data is processed in step 52 to interpret the data and create one or more scores as discussed above. The score is then used in step 54 to decide an appropriate action to take, i.e. the nature and format of the output signal generated by the parent unit.

The generated output may give the parent user feedback on their emotional state or it may inform relatives or friends that one or both of the parents need urgent help. This may be achieved by sending messages over a wide area network, e.g., the internet, to those other individuals using the communications systems as discussed above.

As mentioned above, one option is for all of the monitoring to be performed by the parent unit. However, this would require the parent to be holding the parent unit, which is often not the case.

By making use of external devices, in particular wearables, the system becomes more flexible. Those external devices may for example be used as soon as the baby monitor is switched on. For this purpose, the system will have access to such wearables (by the user giving the permission for the system software to receive data from the wearable and for the wearable to provide data to the system).

From this initial turning on of the baby monitor, e.g. time t=0, changes in emotional state can be assessed. Information can also be derived relating to the interaction with the parent unit and the baby unit, such as:
(i) the baby monitor has moved (using an accelerometer or gyroscope at the baby unit).
(ii) the person wearing the wearable has interacted with the baby monitor (using sensing electrodes, or body area networks, matching accelerometer patterns for the parent unit and the baby unit).
(iii) the length of time during which the person interacts with the baby monitor (time of interaction derived from accelerometer data).
(iv) the type of interaction (e.g. using a talk-back function, lowering the sound level or completely muting the sound).
(v) how often the baby monitor triggers due to noise detection above the threshold (e.g. the baby is crying or talking etc.).

The emotional state monitoring may be triggered rather than operating all the time, based on the various sensing data that has been collected such as described above.

Most simply, data from the monitoring unit may be used only when the baby unit and parent unit are turned on.

In addition, there can also be detection of the parent unit being actively used, for example picked up, or worn or held. This indicates that the parent is concerned about the baby. This can be assessed by an accelerometer forming part of the sensing integrated into the parent unit.

The monitoring unit may also be triggered when the parent unit is sending an alarm (e.g. transmitting sound because a threshold sound level is reached at the baby unit). This is when the parent unit is receiving data (audio and optionally also video) from the baby unit, and thus the parent is aware that the baby is likely not to be sleeping. This is a likely time for a heightened emotion state.

For example, one triggering of the collection of emotional state information may be when either one of the two situations of a baby unit alarm or during active use of the parent unit is present, all while the baby unit is turned on. The system then activates the sensors of the monitoring unit (or requests or processes monitoring unit data from a remote monitoring unit) so that the emotional state of the parent can be determined, using the sensors described above.

In one example, the emotional state is defined based on the combination of facial image data captured by a cameras and using dry electrodes skin conductivity sensing to capture changes in heartrate and the galvanic skin response. The emotional state can then be compared to an average resting emotional state of the parent and where that average state is given a default score (e.g. 0), every change in emotional state ranging from stressed to angry will increase the score, for example reaching a maximum of 10.

Typical signs of an alarming emotional state may be one or more of:
Excessive looking at the video of the baby;
Extreme elevations in heart rate;
Increased respiration rate;
Shaking/trembling when holding the parenting unit;
Elevated voice levels when using a talkback function;
Angry, irritated facial expression.

Each monitored sign may be allocated a score to be compared with a threshold score, so that the sign can be compared with a normal level to determine a raised emotional state.

For instance, a mother may have been trying to get her baby to sleep for some time now, and just as she wants to go to sleep herself again, the baby phone activates because the baby has started crying again. The reactions of the mother to this are measured with the electrodes from her smartwatch and the camera inside the parent unit. An elevation in the heartrate is for example measured and the camera senses an irritated expression.

The system may also include voice recognition, for example to interpret the mother murmuring to herself: “I'm so fed up with this”.

The system has thus obtained multiple indications of an alarming emotional state, thus activating the generation of a suitable output.

The system then determines an action required to mitigate the emotional state and thus lowering the score back towards the minimum. There are several options the system can perform to achieve this goal.

The system, i.e. the parent unit. can give feedback to the parent themselves to make them aware of their current emotional state, and remind them that after they helped the baby they should find ways to calm down in order to look after themselves too.

The system may also make one partner aware when the other seems to be having emotional problems.

The system can suggest an action to lower the emotional score, e.g. doing a physical yoga exercise.

In the first postnatal weeks, the system may even revert to the maternity carer or midwife if severe emotional upset is detected. Similarly, the system may relay the action request to a relative, friend or friendly neighbor who can then help the parent to minimize their emotional score.

The system can also perform multiple such steps if one step is proven to be unsuccessful in lowering the emotional state score.

As explained above, the detection of the emotional state may be triggered based on sensed signals.

A most basic implementation for example involves only monitoring movement of the parent unit after the parent unit and baby unit are turned on (e.g. at the bed-time of the baby). This movement information alone can also be used to determine that there is excessive use of the parent unit (minutes per day), and this information can be compiled into a report.

A more complex triggering also takes account of the times when the baby unit is activated because the baby is making sounds, as explained above. The emotional data can then be flagged if it was collected during times when the baby unit was activated (compared to times when there was parent movement but no baby unit activation).

If emotional state information is collected more continuously, the emotional state before and after a flag indicting that the baby unit is active can also be recorded and compared. Data from wearable sensors can be collected continuously so is available before and after times when the baby unit has activated.

There may for example be collection of some sensor data when the baby monitoring system is turned on (e.g. using external wearables) and then triggering of additional sensor information from the monitoring unit based on the triggers explained above.

In all cases, reports of negative emotional states can be generated.

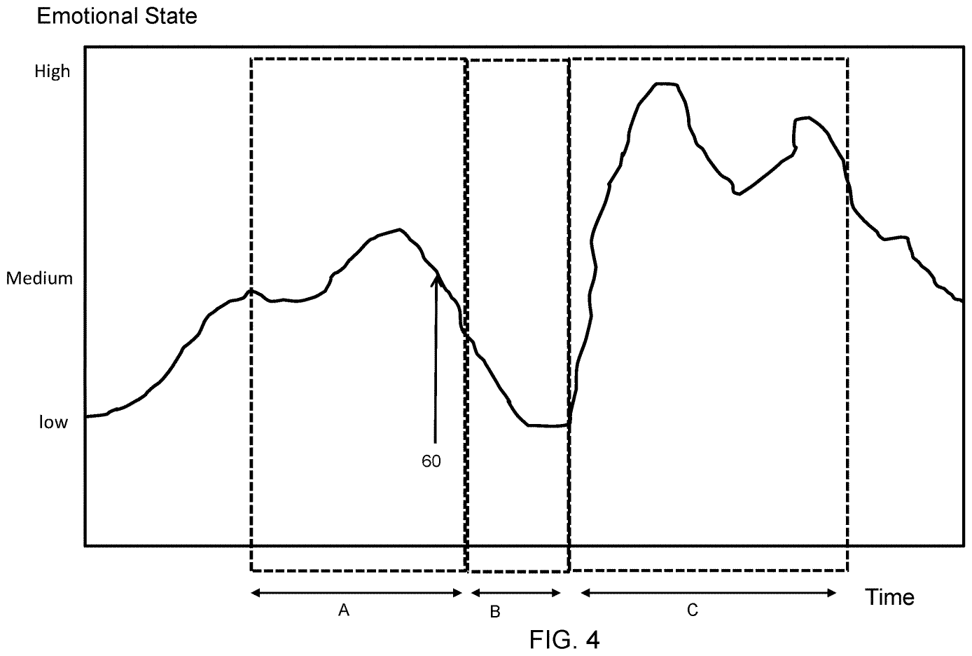

FIG. 4 shows an example of a plot of the emotional state of a parent over time.

The emotional state is for example derived from various sensor data such as the average heart rate, activity levels (e.g. high heart rate combined with low activity is indicative of stress), galvanic skin response and other sensing. Time period A is the bed-time routine, with the baby unit and parent unit turned on at time 60.

During time period B, the baby is quiet and the parents are relaxing downstairs.

During time period C, the baby is crying, and the parents cannot stop it. The baby is finally silent at the end of time period C.

External wearables enable the emotion data to be collected even before time 60, whereas with a system fully integrated into the baby monitor system, the data before time 60 would not be available. Thus, the connection of the system to additional wearable sensors enables the processing of more data.

The reporting generated by the system can take various forms.

One option is simply to report the findings in respect of an individual parent to that individual parent, for the purposes of their self-reflection to the parent.

A second option to share the report between multiple devices/users, e.g. between parents or between direct relatives or guardians.

A third option is to present the report to a third party if it is assessed that professional help is needed or in the case of a single parent there is no possible sharing between multiple devices/users.

As explained above, the system may make use of external sensors, for example existing sensors in mobile phones. The parents for example each have their own mobile phone, and they each connect to the (same) baby monitor system via an app. Wearable sensors of each parent may be paired to their individual phone by the app, and the baby monitoring system requests permission to access that sensor data.

Each parent thus has their own profile, and the external wearables can be linked to that profile. Thus, both parents use their own phone, but a common account links the phones and wearables to the same baby monitor. Of course, access rights and the reporting preferences can be set by the user.

Various systems are known for determining an emotional state from various possible sensing inputs, and indeed any suitable ways of assessing the parent emotional state may be integrated into the system, or accessed by the system (when they are part of a remote device).

The processing to generate the output is for example carried out at the parent unit, but the processing could instead be performed remotely.

Variations to the disclosed embodiments can be understood and effected by those skilled in the art in practicing the claimed invention, from a study of the drawings, the disclosure and the appended claims. In the claims, the word “comprising” does not exclude other elements or steps, and the indefinite article “a” or “an” does not exclude a plurality.

Functions implemented by a processor may be implemented by a single processor or by multiple separate processing units which may together be considered to constitute a “processor”. Such processing units may in some cases be remote from each other and communicate with each other in a wired or wireless manner.

The mere fact that certain measures are recited in mutually different dependent claims does not indicate that a combination of these measures cannot be used to advantage.

A computer program may be stored/distributed on a suitable medium, such as an optical storage medium or a solid-state medium supplied together with or as part of other hardware, but may also be distributed in other forms, such as via the Internet or other wired or wireless telecommunication systems.

If the term “adapted to” is used in the claims or description, it is noted the term “adapted to” is intended to be equivalent to the term “configured to”. If the term “arrangement” is used in the claims or description, it is noted the term “arrangement” is intended to be equivalent to the term “system”, and vice versa.

Any reference signs in the claims should not be construed as limiting the scope.

1. A baby monitoring system comprising:
a baby unit comprising at least a microphone for picking up baby sounds and a communication system;
a parent unit comprising at least a speaker and a communications system, wherein the speaker is for outputting baby sounds transmitted from the baby unit to the parent unit using the communications systems;
a monitoring unit for monitoring a parent emotional state or an input device for receiving information concerning a parent emotional state from a remote monitoring unit, the monitoring unit or remote monitoring unit comprising one or more of a galvanic skin response sensor, a heart rate sensor, a respiration rate sensor, a motion sensor and a camera; and
an output unit for generating an output which depends on the parent emotional state,
wherein the system further comprises a trigger system for triggering the monitoring unit to obtain a parent emotional state when the baby unit triggers the parent unit to activate in order to alert the parent to baby activity or when the parent unit is detected as being actively used.

2. The system of claim 1, comprising a video baby monitoring system, wherein the baby unit comprises a camera and the parent unit comprises a display

3. The system of claim 1, wherein the monitoring unit comprises a galvanic skin response sensor.

4. The system of claim 1, wherein the monitoring unit comprises a heart rate sensor and/or a respiration rate sensor.

5. The system of claim 3, wherein the monitoring unit comprises a dry electrode sensor for monitoring heart rate and/or galvanic skin response.

6. The system of claim 1, wherein the monitoring unit comprises a camera for capturing images of the parent.

7. The system of claim 6, comprising an image processing module for processing images from the camera to detect one or more of:
excessive looking at a video of the baby; and
an angry or irritated facial expression.

8. The system of claim 1, wherein the monitoring unit comprises a motion sensor such as an accelerometer.

9. The system of claim 8, comprising a motion processing module for processing data from the accelerometer to detect parent shaking or trembling.

10. The system of claim 1, wherein the output unit is configured to:
provide feedback to the parent; or
send a communication to a relative, friend, or maternity carer.

11. A baby monitoring method, comprising:
picking up baby sounds at a baby unit of a baby monitoring system;
transmitting baby sounds from the baby unit to a parent unit;
outputting the baby sounds at the parent unit;
monitoring a parent emotional state using a monitoring unit comprising one or more of a galvanic skin response sensor, a respiration rate sensor, a heart rate sensor a motion sensor and a camera, wherein the monitoring of a parent emotional state is triggered when the baby unit triggers the parent unit to activate to alert the parent to baby activity or when the parent unit is detected as being actively used; and
generating an output which depends on the parent emotional state.

12. A computer program comprising computer program code which is adapted, when said program is run on a processor of the system of claim 1.
